(19) United States
(12) Patent Application Publication
(10) Pub. No.: US 2013/0096082 A1
(43) Pub. Date: Apr. 18, 2013

(54) HEMOSTATIC COMPOSITIONS

(71) Applicants: Baxter International Inc., Deerfield, IL (US); Baxter Healthcare S.A., Glattpark (Opfikon) (CH)

(72) Inventors: Katharina Harkamp, Vienna (AT); Katarzyna Gorna, Vienna (AT); Klaus Tschetschkowitsch, Vienna (AT)

(73) Assignees: Baxter Healthcare S.A., Glattpark (Opfikon) (CH); Baxter International Inc., Deerfield, IL (US)

(21) Appl. No.: 13/648,832

(22) Filed: Oct. 10, 2012

Related U.S. Application Data
(60) Provisional application No. 61/545,926, filed on Oct. 11, 2011.
(60) Provisional application No. 61/552,310, filed on Oct. 27, 2011.

(51) Int. Cl. A61K 31/722; A61M 5/19

(52) U.S. Cl. CPC A61K 31/722; A61M 5/19
USPC 514/55; 604/82

(57) ABSTRACT

The invention discloses a hemostatic composition composing chitin or a water insoluble chitin derivative, especially water insoluble chitosan, in particulate form, wherein the composition is present in paste form.

This application claims the benefit of U.S. Ser. No. 61/545,926, filed Oct. 11, 2011 and 61/552,310 filed Oct. 27, 2011, which is incorporated herein by reference.

### FIELD OF THE INVENTION

The present invention relates to hemostatic compositions and processes for making such compositions.

### BACKGROUND OF THE INVENTION

Hemostatic compositions in dry storage-stable form that comprise biocompatible, biodegradable, dry stable granular material are known e.g. from WO98/008550A or WO2003/007845A. These products have been successfully applied on the art for hemostasis. Floseal® is an example for a powerful and versatile hemostatic agent consisting of a granular gelatin matrix swollen in a thrombin-containing solution to form a flowable paste.

Since such products have to be applied to humans, it is necessary to provide highest safety standards for quality, storage-stability and sterility of the final products and the components thereof. On the other hand, manufacturing and handling should be made as convenient and efficient as possible.

Flowable hemostats are used to control moderate, but also frequently happening excessive arterial bleeding. The consistency of flowable hemostats (i.e. solid particles suspended in a hydration medium to obtain a paste-like substance) enables a precise application of the product to the hard-to-reach bleeding sites as e.g. during minimal invasive surgery. The composition of commercially available flowable hemostats is usually based on gelatin used with or without thrombin or collagen with thrombin.

Once of the most effective hemostatic products currently on the market is Floseal® Hemostatic Matrix (Baxter) comprising crosslinked gelatin particles and lyophilized thrombin that prior to applying needs to be reconstituted in calcium chloride solution.

Additionally to gelatin/collagen based biomaterials chitosan is another biopolymer that is used as a hemostatic agent.

Chitosan is a linear polysaccharide composed of randomly distributed β-(1-4)-linked D-glucosamine (deacetylated unit) and N-acetyl-D-glucosamine (acetylated unit). Chitosan is produced commercially by deacetylation of chitin, which is the structural element in the exoskeleton of crustaceans (crabs, shrimp, etc.) and cell walls of fungi. Chitosan is hypoallergenic, and has natural anti-bacterial properties, further supporting its use in field bandages. Currently the product “HemCon Bandage” from the company HemCon Medical Technologies Inc. is licensed in the US for external, temporary control of severely bleeding wounds for emergency cases. Another chitosan-based product for arresting of even lethal bleeding is Celox from MedTrade Products Ltd. Celox™ is made with chitosan that degrades by lysozyme, a human enzyme, to leave glucosamine, a sugar which is normally found in the body. The product is applied in a form of porous granules with very high surface area. When mixed with blood, Celox™ forms a robust gel like clot in 30 seconds. It works independently of the bodies normal clotting processes. A further wound dressing sheet comprising chitosan layers is disclosed in WO02/102276A2. In WO2007/074328A1 a hemostatic powder comprising a chitosan salt is described: the chitosan salt is made by conversion of (water insoluble) chitosan into a water soluble salt form which are described as being “ideally suited for hemostatic application due to its conversion to glucosamine by lysozyme. Blends made of chitosan and gelatin for biomedical applications are disclosed by Chiono et al. (J. Mater. Sci. Mater. Med. 19 (2008), 889-898). However, water insoluble chitosan was described to only absorb platelets but not to be able to cause erythrocyte aggregation (Yang et al.; J. Biomed. Mat. Res. B: Appl. Biomat. (2007), p. 30853). Accordingly, only soluble chitosan (chitosan acetic acid saline solution) was regarded as an effective hemostat.

Thus, in summary chitosan is used as a hemostatic material in its predominantly protonated form (at a low pH value) in which it is soluble in water and forms a sticky adherent hydrogel-like substance upon contact with blood or tissue fluids.

It is an object of the present invention to provide an alternative hemostatic composition to the hemostatic compositions based on a crosslinked gelatin with comparable or improved performance compared to the gelatin products according to the prior art. The compositions should also be provided in a convenient and usable manner, namely as a flowable paste usable in endoscopic surgery and microsurgery. The products should preferably be provided in product formats enabling a convenient provision of “ready-to-use” hemostatic compositions, which can be directly applied to an injury without any time consuming reconstitution steps.

### SUMMARY OF THE INVENTION

Therefore, the present invention provides a hemostatic composition comprising chitin or a water insoluble chitin derivative, especially water insoluble chitosan, in particulate form, wherein the composition is present in paste form.

The invention also refers to the use of this hemostatic composition for treating an injury selected from the group consisting of a wound, a hemorrhage, damaged tissue and/or bleeding tissue comprising administering such a hemostatic composition and kits making such a hemostatic composition for the treatment of such injury.

### DESCRIPTION OF SPECIFIC EMBODIMENTS OF THE INVENTION

The present invention provides a hemostatic composition comprising chitin or a water insoluble chitin derivative, especially water insoluble chitosan, (also referred to hereinafter as the “chitin/chitosan” composition including any other suitable water insoluble chitin derivative) in particulate form, wherein the composition is present in paste form. Surprisingly, it was found that a flowable paste obtained from water insoluble chitosan granules is an effective hemostat. The hemostatic mode of action of water insoluble chitosan powder is different than that of chitosan acetic acid physiological saline solution (Yang et al. 2008). The solid state chitosan does bind platelets but is not able to cause erythrocytes to aggregate or deform as the chitosan acetic acid physiological saline solution does.

Despite these prejudices against hemostatic properties of chitin/water insoluble chitosan, it was surprisingly found that water insoluble chitosan powder formulated as a flowable paste is indeed suitable as a hemostatic agent for diverse kind of bleedings.

Insoluble chitosan according to the present invention is characterized in that a supernatant over a powder form of an insoluble chitosan of the present invention contains less than 5 mg of dissolved solids when measured in a phosphate buffer at physiological pH and 37° C. (11.5 ml buffer over 150 mg chitosan) after 2 hours.

The preferred water insoluble chitin derivative according to the present invention is chitosan. Other suitable examples include hm chitosan (Dowling et al., Biomaterials 32 (2011), 3351-3357). The degree of deacetylation of chitin (% DD) can be determined by NMR (Nuclear magnetic resonance) spectroscopy, and the % DD in commercial chitosans is in the range of 60-100%.

The amino group in chitosan has a pKa value of ˜6.5, thus, chitosan is positively charged and soluble in acidic solution with a charge density dependent on pH and the % DD-value. This makes chitosan a bioadhesive which readily binds to negatively charged surfaces such as mucosal membranes. Chitosan is biocompatible and biodegradable.

The hemostatic composition according to the present invention preferably contains the hemostatic polymer according to the present invention (chitin/chitosan or another water insoluble chitin derivative) and, optionally, e.g. the crosslinked gelatin in particulate form, especially as granular material. This granular material can rapidly swell when exposed to a fluid (i.e. the diluent) and in this swollen form is capable of contributing to a flowable paste that can be applied to a bleeding site. Most of the particles contained in this granular material (e.g. more than 90% w/w) have preferably particle sizes of 10 to 1,000 μm, especially 20 to 700 μm.

Preferably, the water insoluble chitin derivative, especially water insoluble chitosan, is provided in lyophilized form.

The water insoluble chitin/chitin derivative can be applied as the only hemostatic polymer in the hemostatic composition or in combination with another hemostatic polymer.

Such a hemostatic polymer to be provided in combination with chitin/chitosan or its derivatives can be any biocompatible polymer suitable for use in hemostasis. Such biocompatible polymers may be formed from biologic and non-biologic polymers. Preferably, this hemostatic polymer is a protein, a polysaccharide, a biologic polymer, a non-biologic polymer; and derivatives and combinations thereof. Suitable proteins include gelatin, collagen, albumin, hemoglobin, fibrinogen, fibrin, casein, fibronectin, elastin, keratin, and laminin; and derivatives and combinations thereof. Particularly preferred is the use of gelatin or soluble fibrillar or non-fibrillar collagen, more preferably gelatin, and exemplary gelatin formulations are set forth below. Other suitable biologic polymers include polysaccharides, such as glycosaminoglycans, pectins, starch, cellulose, dextran, hemicellulose, xylan, agarose, alginate and chitosan; and derivatives and combinations thereof. Suitable non-biologic polymers will be selected to be degradable by either of two mechanisms, i.e. (1) break down of the polymeric backbone or (2) degradation of side chains which result in aqueous solubility. Exemplary non-biologic biocompatible polymers suitable for use in hemostasis include synthetics, such as polyacrylates, polymethacrylates, polyacrylamides, polymethacrylamides, polyethyleneimines, polyvinyl resins, polylactide-glycolides, polycaprolactones, polyoxyethlenes, polylysine, polyarginine and polyamidoamine (PAMAM) dendrimers; and derivatives and combinations thereof. Also combinations of different kinds of polymers are possible (e.g. proteins with polysaccharides, proteins with non-biologic hydrogel-forming polymers, etc.).

“A derivative thereof” includes any chemically modified polymer, such as e.g. a crosslinked polymer.

According to a preferred embodiment of the present invention, the biocompatible polymer is selected from the group consisting of gelatin, collagen, albumin, fibrinogen, fibrin and derivatives thereof (as defined above); especially preferred the polymer is gelatin or collagen; especially preferred is crosslinked gelatin.

According to a preferred embodiment, the hemostatic composition according to the present invention additionally comprises crosslinked gelatin. Preferably, the crosslinked gelatin is glutaraldehyde-crosslinked gelatin or genipin-crosslinked gelatin, preferably type B gelatin. Examples of suitable gelatin materials for crosslinking are described i.a. in examples 1 and 2 of EP1803417B1, example 14 of U.S. Pat. No. 6,066,325A and U.S. Pat. No. 6,063,061A. Preferably, the biocompatible polymer suitable for use in hemostasis is a gelatin with a Bloom strength of 200 to 400, especially a type B gelatin with a Bloom strength of 200 to 400. Bloom is a test to measure the strength of gelatin. The test determines the weight (in grams) needed by a probe (normally with a diameter of 0.5 inch) to deflect the surface of the gel 4 mm without breaking it. The result is expressed in Bloom (grades). To perform the Bloom test on gelatin, a 6.67% gelatin solution is kept for 17-18 hours at 10° C. prior to being tested. The nature of the gelatin can have advantageous properties on the hemostatic properties of the combined product. Type B gelatin has proven to be specifically advantageous. A specifically preferred gelatin preparation can be prepared by processing young bovine corium with 2 N NaOH for about 1 h at room temperature, neutralizing to pH 7-8, homogenizing and heating to 70° C. The corium is then fully solubilized to gelatin with 3-10% (w/w), preferably 7-10% (w/w) gelatin in solution. This solution can be cast, dried and ground to provide gelatin type B powder.

Gelatin may also be used with processing aids, such as PVP, PEG and/or dextran as re-hydration aids

According to a preferred embodiment, the crosslinked gelatin is provided from a dry crosslinked gelatin. This dry crosslinked gelatin powder can be prepared to re-hydrate rapidly if contacted with a pharmaceutically acceptable diluent. The gelatin granules, especially in the form of a gelatin powder, preferably comprise relatively large particles, also referred to as fragments or sub-units, as described in WO98/08550A and WO2003/007845A. A preferred (median) particle size will be the range from 10 to 1,000 μm, preferably from 200 to 800 μm, but particle sizes outside of this preferred range may find use in many circumstances. The dry compositions will also display a significant “equilibrium swell” when exposed to an aqueous re-hydrating medium (=diluents, also referred to as reconstitution medium or re-hydration medium). Preferably, the swell will be in the range from 500% to 1000%. “Equilibrium swell” may be determined by subtracting the dry weight of the gelatin hydrogel powder from its weight when fully hydrated and thus fully swelled. The difference is then divided by the dry weight and multiplied by 100 to give the measure of swelling. The dry weight should be measured after exposure of the material to an elevated temperature for a time sufficient to remove substantially all residual moisture, after two hours at 120° C. The equilibrium hydration of the material can be achieved by immersing the dry material in a pharmaceutically acceptable diluent, such as aqueous saline, for a time period sufficient for the water content to become constant, typically for from 18 to 24 hours at room temperature.

Exemplary methods for producing crosslinked gelatins are as follows. Gelatin is obtained and suspended in an aqueous solution to form a non-crosslinked hydrogel, typically having a solids content from 1% to 70% by weight, usually from 3% to 10% by weight.

In one particular aspect of the present invention, compositions will comprise crosslinked gelatin powders having a moisture content of 20% (w/w) or less, wherein the powder was crosslinked in the presence of a re-hydration aid so that the powder has an aqueous re-hydration rate which is at least 5% higher than the re-hydration rate of a similar powder prepared without the re-hydration aid. The “re-hydration rate” is defined according to EP1803417B1 to mean the quantity of an aqueous solution, typically 0.9% (w/w) saline, that is absorbed by a gram of the powder (dry weight basis) within thirty seconds, expressed as g/g: The rehydration rate is measured by mixing the crosslinked gelatin with saline solution for 30 seconds and depositing the wet gelatin on a filter membrane under vacuum to remove the free aqueous solution. One then records the weight of the wet gelatin retained on the filter, dries it (e.g. 2 h at 120° C), then records the dry weight of the gelatin and calculates the weight of solution that was absorbed per gram of dry gelatin.

Preferred compositions of the present invention will have a re-hydration rate of at least 3 g/g, preferably at least 3.5 g/g, and often 3.75 g/g or higher. Re-hydration rates of similar powders prepared without the re-hydration aids are typically below three, and a percentage increase in re-hydration rate will usually be at least 5%, preferably being at least 10%, and more preferably being at least 25% or higher.

Suitably mixing ratios of chitin/chitosan with other hemostatic polymers may be established depending on the nature of the other polymer(s) in the paste form. Preferred combination products containing mixtures of chitin or a water insoluble chitin derivative, especially water insoluble chitosan, with crosslinked gelatin have a ratio (in % (w/w)) of 5:95 to 80:20, preferably 10:90 to 50:50, especially 20:80 to 40:60. A preferred composition comprises 30% chitosan and 70% crosslinked gelatin. However, for certain purposes and to achieve other product characteristics, combinations with more chitin/chitosan may be used, e.g. a mixture of 60 to 80% chitin/chitosan with 40 to 20% crosslinked gelatin, especially a formulation with 70% chitosan and 30% crosslinked gelatin.

According to a preferred embodiment, the hemostatic composition according to the present invention contains NaCl, CaCl₂, sodium acetate and/or thrombin, preferably 10 to 1000 I.U. thrombin/ml, especially 250 to 700 I.U. thrombin/ml.

According to another aspect, the present invention relates to a hemostatic composition according to the present invention for use in the treatment of an injury selected from the group consisting of a wound, a hemorrhage, damaged tissue, bleeding tissue and/or bone defects.

Another aspect of the present invention is a method of treating an injury selected from the group consisting of a wound, a hemorrhage, damaged tissue and/or bleeding tissue comprising administering a hemostatic composition according to the present invention to the site of injury.

According to another aspect, the present invention also provides a method for delivering the hemostatic composition according to the invention to a target site in a patient's body, said method comprising delivering a hemostatic composition produced by the process according to the present invention to the target site. Although also the dry composition can be directly applied to the target site (and, optionally be contacted with a pharmaceutically acceptable diluent a the target site, if necessary), it is preferred to contact the dry hemostatic composition with a pharmaceutically acceptable diluent before administration to the target site, so as to obtain a hemostatic composition according to the present invention in paste form.

In such a method, a kit for making a flowable paste of chitin or a water insoluble chitin derivative, especially water insoluble chitosan, for the treatment of an injury selected from the group consisting of a wound, a hemorrhage, damaged tissue and/or bleeding tissue, may be applied, this kit comprising
a) a dry hemostatic composition comprising chitin or a water insoluble chitin derivative, especially water insoluble chitosan, and
b) a pharmaceutically acceptable diluent for reconstitution of the hemostatic composition.

The crosslinked gelatin component of the kit according to the present invention is preferably provided as a dry composition, wherein chitin or a water insoluble chitin derivative, especially water insoluble chitosan, is present in dry form.

A “dry” chitin or water insoluble chitin derivative, especially water insoluble chitosan, composition according to the present invention has only a residual content of moisture. Usually, the dry composition according to the present invention has a residual moisture content below these products, preferably below 15% moisture, more preferred below 10% moisture, more preferred below 5%, especially below 1% moisture. The chitin or water insoluble chitin derivative, especially water insoluble chitosan, component of the kit according to the present invention can also have lower moisture content, e.g. 0.1% or even below. Preferred moisture contents of the chitin or water insoluble chitin derivative, especially water insoluble chitosan, component of the kit according to the present invention are 0.1 to 10%, especially 0.5 to 5%. It is clear that the dryer the composition is, the longer their shelf life is and the lower is the risk that the hemostatic properties of the composition as a whole suffer.

The dry chitin or a water insoluble chitin derivative, especially water insoluble chitosan, in particulate form (as well as other polymers, especially crosslinked gelatin suitable for use in hemostasis) in the kit according to the present invention is preferably provided in powder form, especially wherein the powder particles have a median particle size of 10 to 1000 μm. A “dry granular preparation of crosslinked gelatin” optionally admixed to chitosan/chitin according to the present invention is in principle known e.g. from WO 98/08550 A; accordingly, the drying and granulation methods known for e.g. glutaraldehyde-crosslinked gelatin may also be applied for the genipin-crosslinked material, especially gelatin. Preferably, the crosslinked gelatin is therefore a biocompatible, biodegradable dry stable granular material.

The dry particles in the kit according to the present invention is usually provided with particle sizes of 10 to 1,000 μm. Usually, the gelatin particles have a mean particle diameter (“mean particle diameter” is the median size as measured by laser diffractometry; “median size” (or mass median particle diameter) is the particle diameter that divides the frequency distribution in half; fifty percent of the particles of a given preparation have a larger diameter, and fifty percent of the particles have a smaller diameter) from 10 to 1000 μm, especially 50to 700 μm (median size). Applying larger particles is mainly dependent on the medical necessities; particles with smaller mean particle diameters are often more difficult to handle in the production process. The dry particles are therefore provided in granular form. Although the terms powder and granular (or granulates) are sometimes used to distinguish separate classes of material, powders are defined herein as a special sub-class of granular materials. In particular, powders refer to those granular materials that have the finer grain sizes, and that therefore have a greater tendency to form clumps when flowing. Granules include coarser granular materials that do not tend to form clumps except when wet. For the present application the particles used are those which can be coated by suitable coating techniques Particle size of the polymer granules according to the present invention cars therefore easily be adapted and optimized to a certain coating technique by the necessities of this technique.

The present composition in particulate form suitable for use in hemostasis may include dimensionally isotropic or non-isotropic forms. For example, the chitin/chitosan component in the kit according to the present invention may be granules or fibers; and may be present in discontinuous structures, for example in powder forms.

The chitin/chitosan component (including, optionally, further hemostatic polymers, especially gelatin or oxidized cellulose) of the kit according to the present invention is liquid absorbing. For example, upon contact with liquids, e.g. aqueous solutions or suspensions (especially a buffer or blood) the chitin/chitosan takes up the liquid and will display a degree of swelling, depending on the extent of hydration. The material preferably absorbs from at least 100%, preferably about 400% to about 2000%, especially from about 500% to about 1300% water or aqueous buffer by weight, corresponding to a nominal increase in diameter or width of an individual particle of subunit in the range from e.g. approximately 50% to approximately 500%, usually from approximately 50% to approximately 250%. For example, if the (dry) granular particles have a preferred size range of 0.01 mm to 1.5 mm, especially of 0.05 mm to 1 mm, the fully hydrated composition (e.g. after administration on a wound or after contact with an aqueous buffer solution) may have a size range of 0.05 mm to 3 mm, especially of 0.25 mm to 1.5 mm.

The equilibrium swell of the chitin/chitosan component in the kit of the present invention optionally comprising e.g. crosslinked gelatin may generally range e.g. from 100% to 1300%, preferably being from 300% to 1100%, especially from 500% to 900%, depending on its intended use. Such equilibrium swell (for a crosslinked polymer such as gelatin) may be controlled e.g. by varying the degree of crosslinking, which in turn is achieved by varying the crosslinking conditions, such as the duration of exposure of a crosslinking agent, concentration of a crosslinking agent, crosslinking temperature, and the like. Materials having differing equilibrium swell values perform differently in different applications. For example moderate to mild bleedings as e.g. observed in the liver incision model may need a swell in the range of 700% to 950%, whereas more severe bleedings (such as e.g. arterial or spurting bleeding) may need a material with lower equilibrium swell values (e.g. 500%-600%). Thus, the ability to control crosslinking and equilibrium swell allows the compositions of the present invention to be optimized for a variety of uses. In addition to equilibrium swell, it is also important to control the hydration of the material immediately prior to delivery to a target site. Hydration and equilibrium swell are, of course, intimately connected. A material with 0% hydration will be non-swollen. A material with 100% hydration will be at its equilibrium water content. Hydrations between 0% and 100% will correspond to swelling between the minimum and maximum amounts.

A preferred further component of such a kit is—specifically if the hemostatic composition is contained in dry form—a pharmaceutically acceptable diluent for reconstitution of the hemostatic composition. Further components of the kit may be administration means, such as syringes, catheters, brushes, etc. (if the compositions are not already provided in the administration means) or other components necessary for use in medical (surgical) practice, such as substitute needles or catheters, extra vials or further wound cover means. Preferably, the kit according to the present invention comprises a syringe housing the dry and stable hemostatic composition and a syringe containing the diluent (or provided to take up the diluent from another diluent container). Preferably, these two syringes are provided in a form adapted to each other so that the diluent can be delivered to the dry hemostatic composition by another entry than the outlet for administering the reconstituted composition.

For example, this diluent may contain a substance selected from the group consisting of NaCl, CaCl₂ and sodium acetate. The diluent preferably comprises a buffer or buffer system, preferably at a pH of 3.0 to 10.0. For example, a pharmaceutically acceptable diluent comprises water for injection, and—independently of each other—50 to 200 mM NaCl (preferably 150 mM), 10 to 80 mM CaCl₂ (preferably 40 mM) and 1 to 50 mM sodium acetate (preferably 20 mM). Preferably, the diluent can also include a buffer or buffer system so as to buffer the pH of the reconstituted dry composition, preferably at a pH of 3.0 to 10.0, more preferred of 6.4 to 7.5, especially at a pH of 6.9 to 7.1.

These aqueous solutions may further contain other ingredients, such as excipients. An “excipient” is an inert substance which is added to the solution, e.g. to ensure that thrombin retains its chemical stability and biological activity upon storage (or sterilization (e.g. by irradiation)), or for aesthetic reasons e.g. color. A preferred excipient includes human albumin. Preferred concentrations of human albumin in the reconstituted product are from 0.1 to 100 mg/ml, preferably from 1 to 10 mg/m.

According to a preferred embodiment, the diluent further comprises thrombin, preferably 10 to 1000 I.U. thrombin/ml, especially 250 to 700 I.U. thrombin/ml. Preferably, the hemostatic composition in this ready to use form contains 10 to 100,000 International Units (I.U.) of thrombin, more preferred 100 to 10,000 I.U., especially 500 to 5,000 I.U. The thrombin concentration in the ready-to-use composition is preferably in the range of 10 to 10,000 I.U., more preferred of 50 to 5,000 I.U., especially of 100 to 1,000 I.U./ml. The diluent is used in an amount to achieve the desired end-concentration in the ready-to-use composition. The thrombin preparation may contain other useful component, such as ions, buffers, excipients, stabilizers, etc.

Thrombin (or any other coagulation inducing agent, such as a snake venom, a platelet activator, a thrombin receptor activating peptide and a fibrinogen precipitating agent) can be derived from any thrombin preparation which is suitable for use in humans (i.e. pharmaceutically acceptable). Suitable sources of thrombin include human or bovine blood, plasma or serum (thrombin of other animal sources can be applied if no adverse immune reactions are expected) and thrombin of recombinant origin (e.g. human recombinant thrombin); autologous human thrombin can be preferred for some applications.

In a preferred embodiment, the pharmaceutical acceptable diluent is provided in a separate container. This can preferably be a syringe. The diluent in the syringe can then easily be applied to the final container for reconstitution of the dry hemostatic compositions according to the present invention. If the final container is also a syringe, both syringes can be finished together in a pack. It is therefore preferred to provide the dry hemostatic compositions according to the present invention in a syringe which is finished with a diluent syringe with a pharmaceutically acceptable diluent for reconstituting said dry and stable hemostatic composition.

According to a preferred embodiment, the final container further contains an amount of a stabilizer effective to inhibit modification of the polymer when exposed to the sterilizing radiation, preferably ascorbic acid, sodium ascorbate, other salts of ascorbic add, or an antioxidant.

With such a diluent, a ready to use form of the present hemostatic composition may be provided which can then be directly applied to the patient. Accordingly, also method for providing a ready to use form of a hemostatic composition according to the present invention is provided, wherein the hemostatic composition is provided in a first syringe and a pharmaceutically acceptable diluent for reconstitution is provided in a second syringe, the first and the second syringe are connected to each other, and the fluid is brought into the first syringe to produce a flowable form of the hemostatic composition; and optionally returning the flowable form of the hemostatic composition to the second syringe at least once. Preferably, the ready-to use preparations are present or provided as hydrogels. Products of this kind are known in principle in the art, yet in a different format. Therefore, a method for providing a ready to use form of a hemostatic composition according to the present invention, wherein the hemostatic composition is provided in a first syringe and a pharmaceutically acceptable diluent for reconstitution is provided in a second syringe, the first and the second syringe are connected to each other, and the diluent is brought into the first syringe to produce a flowable form of the hemostatic composition; and optionally returning the flowable form of the hemostatic composition to the second syringe at least once, is a preferred embodiment of the present invention. This process (also referred to as “swooshing”) provides a suitable ready-to-use form of the compositions according to the present invention which can easily and efficiently be made also within short times, e.g. in emergency situations during surgery. This flowable form of the hemostatic composition provided by such a method is specifically suitable for use in the treatment of an injury selected from the group consisting of a wound, a hemorrhage, damaged tissue, bleeding tissue and/or bone defects.

For stability reasons, such products (as well as the products according to the present invention) are usually provided in a dry form in a final container and brought into the ready-to-use form (which is usually in the form of a (hydro-)gel, suspension or solution) immediately before use, necessitating the addition of wetting or solvation (suspension) agents.

According to another aspect, the present invention relates to a method for providing a ready to use form of a hemostatic composition according to the present invention, wherein the hemostatic composition is provided in a first syringe and a diluent for reconstitution is provided in a second syringe, the first and the second syringe are connected to each other, and the fluid is brought into the first syringe to produce a flowable form of the hemostatic composition; and optionally returning the flowable form of the hemostatic composition to the second syringe at least once. Preferably, the flowable form contains chitin/chitosan (preferably chitosan), optionally combined with another hemostatic polymer (preferably crosslinked gelatin), in an amount of 5 to 30% (w/w), preferably of 10 to 25% (w/w), especially of 12 to 20% (w/w).

Preferably, the flowable form of the hemostatic composition according to the present invention contains more than 50% (w/w) particles with a size of 100 to 1000 μm, preferably more than 80% (w/w) particles with a size of 100 to 1000 μm.

The biocompatible hemostatic crosslinked polymer according to the present invention—once applied to a wound—forms an efficient matrix which can form a barrier for blood flow. Specifically the swelling properties of the hemostatic polymer can make it an effective mechanical barrier against bleeding and rebleeding processes.

The present composition may additionally contain a hydrophilic polymeric component (also referred to as “reactive hydrophilic component” or “hydrophilic (polymeric) crosslinker”) which further enhances the adhesion propertied of the present composition. This hydrophilic polymeric component of the hemostatic composition according to the present invention acts as a hydrophilic crosslinker which is able to react with its reactive groups once the hemostatic composition is applied to a patient (e.g. to a wound of a patient or another place where the patient is in need of a hemostatic activity). Therefore it is important for the present invention that the reactive groups of the hydrophilic polymeric component are reactive when applied to the patient. It is therefore necessary to manufacture the hemostatic composition according to the present invention so that the reactive groups of the polymeric component which should react once they are applied to a wound are retained during the manufacturing process.

This can be done in various ways. For example, usual hydrophilic polymeric components have reactive groups which are susceptible to hydrolysis after contact with water. Accordingly, premature contact with water or aqueous liquids has to be prevented before administration of the hemostatic composition to the patient, especially during manufacture. However, processing of the hydrophilic polymeric component during manufacturing may be possible also in an aqueous medium at conditions where the reactions of the reactive groups are inhibited (e.g. at a low pH). If the hydrophilic polymeric components can be melted, the melted hydrophilic polymeric components can be sprayed or printed onto the matrix of the hemostatic composition. It is also possible to mix a dry form (e.g. a powder) of the hydrophilic polymeric component with a dry form of the hemostatic composition. If necessary, then an increase of the temperature can be applied to melt the sprinkled hydrophilic polymeric component to the hemostatic composition to achieve a permanent coating of the hemostatic composition. Alternatively, these hydrophilic polymeric components can be taken up into inert organic solvents (inert vis-à-vis the reactive groups of the hydrophilic polymeric components) and brought onto the matrix of the hemostatic composition. Examples of such organic solvents are dry ethanol, dry acetone or dry dichloromethane (which are e.g. inert for hydrophilic polymeric components, such as NHS-ester substituted PEGs).

In a preferred embodiment the hydrophilic polymer component is a single hydrophilic polymer component and is a polyalkylene oxide polymer, preferably a PEG comprising polymer. The reactive groups of this reactive polymer are preferably electrophilic groups. Alternatively, nucleophilic groups may also be added (e.g. PEG-SH).

The reactive hydrophilic component may be a multi-electrophilic polyalkylene oxide polymer, e.g. a multi-electrophilic PEG. The reactive hydrophilic component can include two or more electrophilic groups, preferably a PEG comprising two or more reactive groups selected from succinimidylesters (—CON(COCH₂)₂), aldehydes (—CHO) and isocyanates (—N═C═O), e.g. a component as disclosed in the WO2008/016983 A (incorporated herein by reference in its entirety) and one of the components of the commercially available ones under the trademark CoSeal®.

Preferred electrophilic groups of the hydrophilic polymeric crosslinker according to the present invention are groups reactive to the amino-, carboxy-, thiol- and hydroxy-groups of proteins, or mixtures thereof.

Preferred amino group-specific reactive groups are NHS-ester groups, imidoester groups, aldehyde-groups, carboxy-groups in the presence of carbodiimides, isocyanates, or THPP (beta-[Tris(hydroxymethyl)phosphino] propionic acid), especially preferred is Pentaerythritolpoly(ethyleneglycol)ether tetrasuccinimidyl glutarate (=Pentaerythritol tetrakis[1-1′-oxo-5′-succinimidylpentanoate-2-poly-oxoethyleneglycole]ether (=an NHS-PEG with MW 10,000).

Preferred carboxy-group specific reactive groups are amino-groups in the presence of carbodiimides.

Preferred thiol group-specific reactive groups are maleimides or haloacetyls.

Preferred hydroxy group-specific reactive group is the isocyanate group. The reactive groups on the hydrophilic crosslinker may be identical (homo-functional) or different (hetero-functional). The hydrophilic polymeric component can have two reactive groups (homo-bifunctional or heterobifunctional) or more (homo/hetero-trifunctional or more).

In special embodiments the material is a synthetic polymer, preferably comprising PEG. The polymer can be a derivative of PEG comprising active side groups suitable for crosslinking and adherence to a tissue.

By the reactive groups the hydrophilic reactive polymer has the ability to crosslink blood proteins and also tissue surface proteins. Crosslinking to the chitin/chitosan is also possible.

The multi-electrophilic polyalkylene oxide may include two or more succinimidyl groups. The multi-electrophilic polyalkylene oxide may include two or more maleimidyl groups.

Preferably, the multi-electrophilic polyalkylene oxide is a polyethylene glycol or a derivative thereof.

In a most preferred embodiment the hydrophilic polymeric component is pentaerythritolpoly(ethyleneglycol)ether tetrasuccinimidyl glutarate (═COH102, also pentaerythritol tetrakis[1-1′-oxo-5′-succinimidylpentanoate-2-poly-oxoethyleneglycole]ether).

The hydrophilic polymeric component is a hydrophilic crosslinker. According to a preferred embodiment, this crosslinker has more than two reactive groups for crosslinking (“arms”), for example three, four, five, six, seven, eight, or more arms with reactive groups for crosslinking. For example, NHS-PEG-NHS is an effective hydrophilic crosslinker according to the present invention. However, for some embodiments, a 4-arm polymer (e.g. 4-arms-p-NP-PEG) may be more preferred; based on the same rationale, an 8-arm polymer (e.g. 8-arms-NHS-PEG) may even be more preferred for those embodiments where multi-reactive crosslinking is beneficial. Moreover, the hydrophilic crosslinker is a polymer, i.e. a large molecule (macromolecule) composed of repeating structural units which are typically connected by covalent chemical bonds. The hydrophilic polymer component should have a molecular weight of at least 1000 Da (to properly serve as crosslinker in the hemostatic composition according to the present invention); preferably the crosslinking polymers according to the present invention has a molecular weight of at least 5000 Da, especially of at least 8000 Da.

For some hydrophilic crosslinkers, the presence of basic reaction conditions (e.g. at the administration site) is preferred or necessary for functional performance (e.g. for a faster crosslinking reaction at the administration site). For example, carbonate or bicarbonate ions (e.g. as a buffer with a pH of 7.6 or above, preferably of 8.0 or above, especially of 8.3 and above) may be additionally provided at the site of administration (e.g. as a buffer solution or as a fabric or pad soaked with such a buffer), so as to allow an improved performance of the hemostatic composition according to the present invention or to allow efficient use as a hemostatic and/or wound adherent material.

The reactivity of the hydrophilic polymeric component (which, as mentioned, acts as a crosslinker) in the composition according to the present invention is retained in the composition. This means that the reactive groups of the crosslinker have not yet reacted with the hemostatic composition and are not hydrolyzed by water (or at least not in a significant amount which has negative consequences on the hemostatic functionality of the present compositions). This can be achieved by combining the hemostatic composition according to the present invention with the hydrophilic crosslinker in a way which does not lead to reaction of the reactive groups of the crosslinker with the hemostatic polymer or with water. Usually, this includes the omitting of aqueous conditions (or wetting), especially wetting without the presence of acidic conditions (if crosslinkers are not reactive under acidic conditions). This allows the provision of reactive hemostatic materials.

Preferred ratios of the hemostatic composition according to the present invention to hydrophilic polymeric component in the hemostatic composition according to the present invention are from 0.1 to 50% w/w, preferably from 5 to 40% w/w.

Whereas for certain uses, the above mentioned hydrophilic polymeric compounds are specifically preferred, there are other uses where the presence of such hydrophilic polymeric compounds is less advantageous. In such cases it may also be preferred to prevent the presence of one such hydrophilic polymeric component comprising reactive groups in a product comprising a biocompatible polymer in particulate form suitable for use in hemostasis, especially if the product is present in paste form.

According to such an embodiment, a product produced by the following process for making a dry and stable hemostatic composition shall be excluded: A process comprising the steps of:
a) providing a first component comprising a dry preparation of a coagulation inducing agent,
b) providing a second component comprising a dry preparation of a biocompatible polymer suitable for use in hemostasis,
c) mixing said first component and said second component under conditions effective to form a wet paste while essentially preventing degradation of the second component by said first component in a final container or transferring said wet paste into a final container,
d) freezing and lyophilizing said paste in said container thereby obtaining a dry and stable hemostatic composition comprising said first and said second component in lyophilized form, and
e) finishing said dry and stable hemostatic composition in said final container to a storable pharmaceutical device containing said first component and said second component in a combined form as a dry and stable hemostatic composition.

Further components may be present in the hemostatic composition according to the present invention. According to preferred embodiments, the hemostatic compositions according to the present invention may further comprise a substance selected from the group consisting of antifibrinolytlc, procoagulant, platelet activator, antibiotic, vasoconstrictor, dye, growth factors, bone morphogenetic proteins and pain killers.

The hemostatic composition according to the present invention may comprise a composition of hemostatic composition according to the present invention and a polyvalent nucelophilic substance, preferably human serum albumin, optionally at a basic pH (e.g. pH 8 to 11, preferably 9 to 10, especially at a pH of 9.5).

The present invention also refers to a finished final container containing the hemostatic composition according to the present invention. This finished container contains the hemostatic composition according to the present invention in a sterile, storage-stable and marketable form. The final container can be any container suitable for housing (and storing) pharmaceutically administrable compounds. Syringes, vials, tubes, etc. can be used; however, providing the hemostatic compositions according to the present invention in a syringe is specifically preferred. Syringes have been a preferred administration means for hemostatic compositions as disclosed in the prior art also because of the handling advantages of syringes in medical practice. The compositions may then preferably be applied (after reconstitution) via specific needles of the syringe or via suitable catheters. The reconstituted hemostatic compositions (which are preferably reconstituted to form a hydrogel) may also be applied by various other means e.g. by a spatula, a brush, a spray, manually by pressure, or by any other conventional technique. Administration of the reconstituted hemostatic composition to a patient by spraying is specifically preferred. Usually, the reconstituted hemostatic compositions according to the present invention will be applied using a syringe or similar applicator capable of extruding the reconstituted composition through an orifice, aperture, needle, tube, or other. Mechanical disruption of the compositions where gelatin is present in gel form can be performed by extrusion through an orifice in the syringe or other applicator, typically having a size in the range from 0.01 mm to 5.0 mm, preferably 0.5 mm to 2.5 mm. Preferably, however, the hemostatic composition will be initially prepared from a dry form having a desired particle size (which upon reconstitution, especially by hydration, yields subunits of the requisite size (e.g. hydrogel subunits)) or will be partially or entirely mechanically disrupted to the requisite size prior to a final extrusion or other application step. If is, of course evident, that these mechanical components have to be provided in sterile form (inside and outside) in order to fulfill safety requirements for human use.

Another aspect of the invention concerns a method for providing a ready-to-use hemostatic composition comprising contacting a hemostatic composition according to the present invention.

The invention is further described in the examples below and the drawing figures, yet without being restricted thereto.

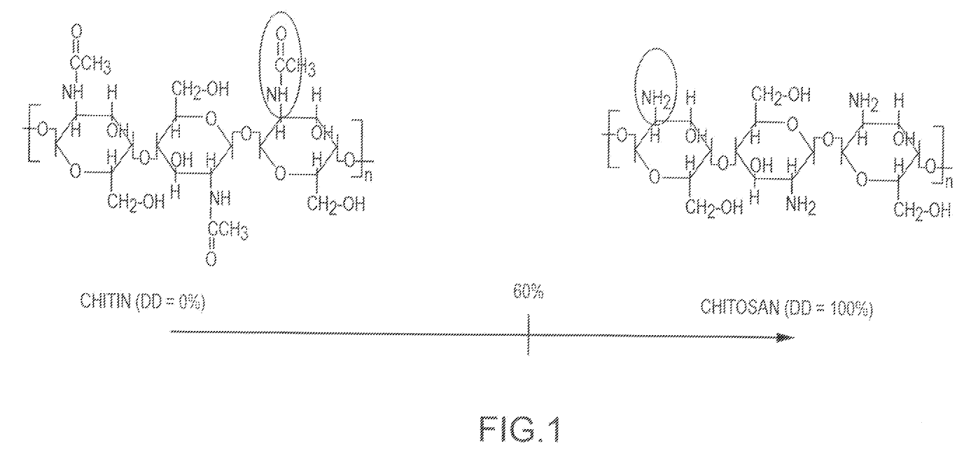

FIG. 1 shows the structure of chitin and chitosan. The arrow below depicts the degree of acetylation of commercially available chitosan products).

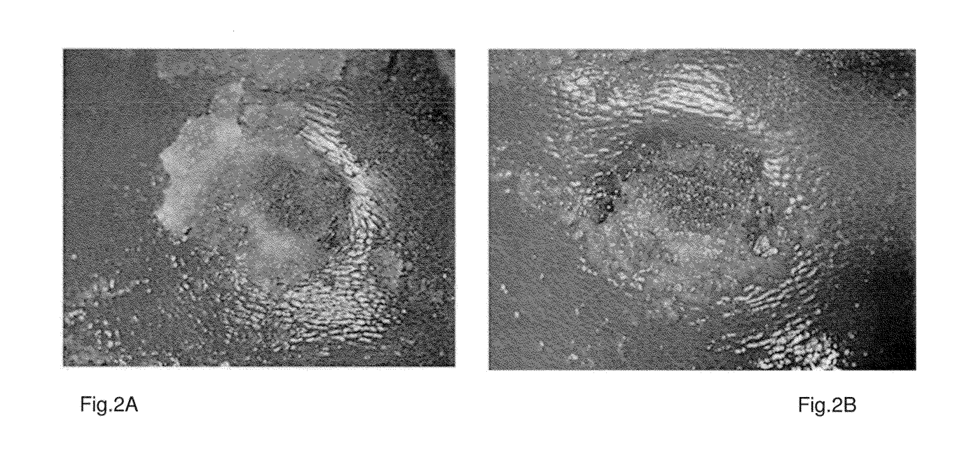

FIG. 2 shows pictures of the liver defect filled with the formulation 0.27 g gelatin-chitosan mixture/ml 40 mM CaCl₂-solution (the gelatin-chitosan mixture contained 27% w/w chitosan) a) after 1 min of approximation and b) after irrigation that was done 3 min post application.

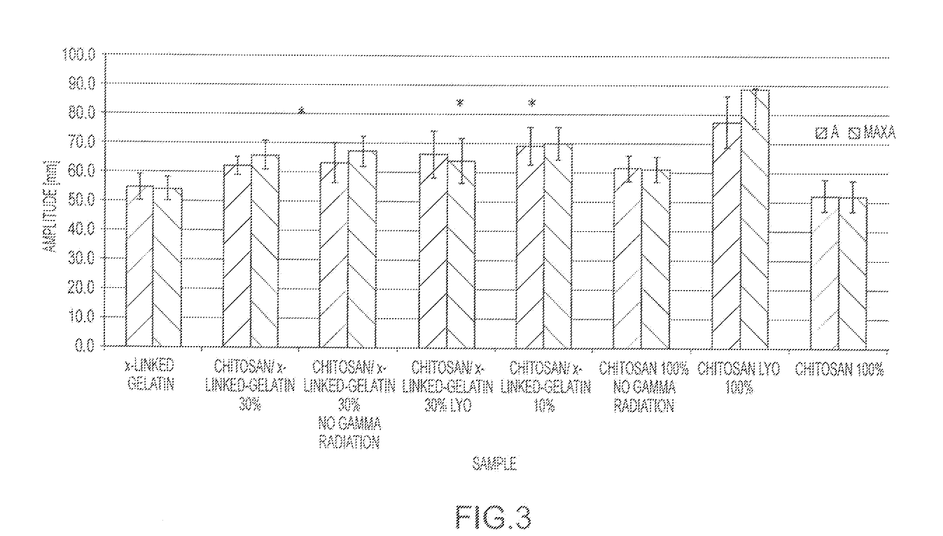

FIG. 3 shows the test results of TEG measures with whole blood of glutaraldehyde crosslinked gelatin; lyophilized and native chitosan and mixtures thereof. All of the samples were gamma irradiated except if Indicated otherwise.

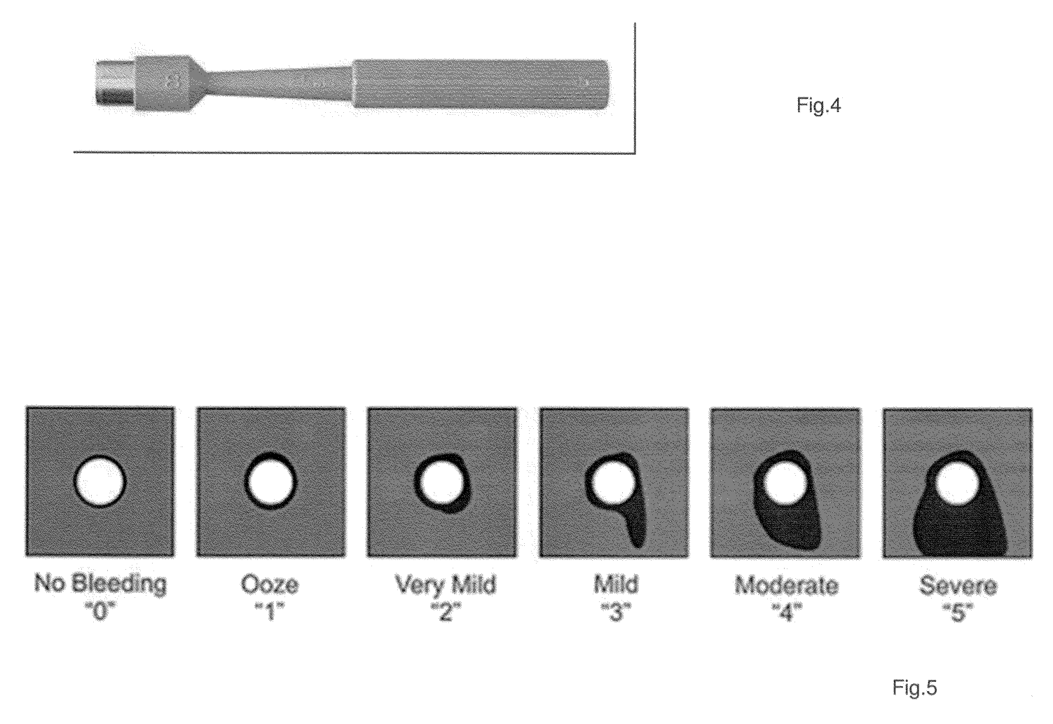

FIG. 4 shows the biopsy punch device used in the animal model.

FIG. 5 shows the degree of bleeding scale scores in the animal model. Bleeding score: 0: no bleeding (product saturated with blood); 1: ooze (blood out of product but no blood drop); 2: very mild (blood drop on the product); 3: mild (blood drop streams down); 4: moderate (small amount of blood streams down); 5: severe (large amount of blood streams down).

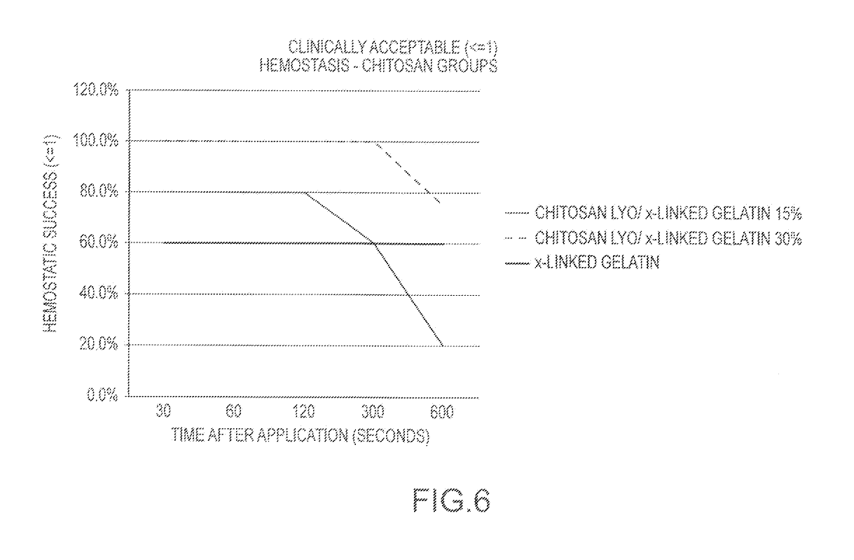

FIG. 6 shows hemostatic efficacy over time measured in a pig liver punch model.

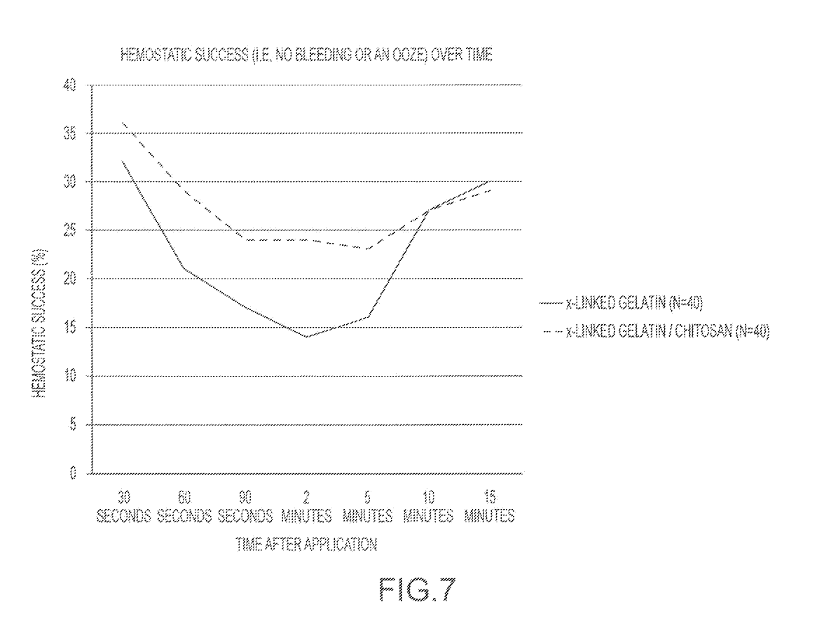

FIG. 7 shows hemostatic success over time in a liver punch model.

### EXAMPLES

### Example 1

### Chitosan Mixed with Dry Crosslinked Gelatin Particles Hydrated with 40 mM CaCl₂ Solution

The product was prepared as follows: 4 g of crosslinked gelatin particles (as prepared according to e.g. EP1803417B1) was mixed with 1.6 g of chitosan (Sigma, product #48165, lot #0001440883) in 50 ml Falcon test tube using end-over-end mixer. After 20 min of mixing, 0.8 g of product was weighted in Floseal syringe. As a diluent 40 mM of CaCl₂ was used in an amount of 3 ml.

The both components (powder and diluent) are mixed by swooshing by pushing content of syringe 21 times back and forth. The material was applied into wound after 2 min of pre-hydration.

### Surgical Procedure:

The test was performed on a heparinized animal (pig). An incision lesion on the exposed lobe of liver was made (see FIG. 1) using the tool presented in FIG. 4. Each lesion in the series was topically treated with not controlled amount of the product. Moistened gauze was used to help approximate the test product to the lesion. Chitosan mixed with dry crosslinked gelatin particles hydrated with 40 mM calcium chloride solution.

### Results:

The results are depicted in FIG. 1. Treatment of the liver defect filled with the formulation 0.27 g gelatin-chitosan mixture/ml 40 mM CaCl₂-solution (containing 27% w/w chitosan) shows that hemostasis was achieved (FIG. 2A: after 1 min of approximation; FIG. 2B: after irrigation that was done 3 mm post application).

### Example 2

### Flowable Hemostatic Containing Mixtures of Glutaraldehyde Crosslinked Gelatin and Solid State Chitosan

In example 1, water insoluble chitosan showed promising results in hemostasis in principle. Therefore, various water insoluble chitosan preparations with different degrees of deacetylation as well as molecular weights were used in order to prepare a flowable hemostatic paste (similar to the hemostatic paste based on crosslinked gelatin according to EP1803417B1) containing varying amounts of chitosan.

There were two distinct groups of variant that were further optimized:
crosslinked gelatin that contained varying amounts of “native” chitosan powder as obtained by the supplier in a particulate form
crosslinked gelatin that contained varying amounts of lyophilized chitosan powder in particulate form. It was anticipated that freeze-drying generally would increase the surface area and could thus potentially lead to better blood uptake of the powder. Therefore a freeze-drying process was developed and the product variants were tested in in vitro as well as in vivo models.

### Example 2.1

### In Vitro Results on Glutaraldehyde Crosslinked Gelatin-Chitosan Mixtures Hydrated with Thrombin Solution

### Thromboelastography Tests:

Glutaraldehyde crosslinked gelatin was mixed with either native chitosan powder or chitosan powder that was obtained after freeze-drying. The samples were subjected to thromboelastography.

### Thromboelastography (TEG):

TEG was performed as follows: A TEG® 5000 Thromboelastography® Hemostasis System was used employing software TEG Analytical Software (TAS) Version 4.2.3. In brief, 0.125 g of test article is reconstituted with 625 μL of the thrombin stock solution (containing 500 IU/ml; 40 mM CaCl₂) by transferring the materials from one syringe to the other at least 20 times (i.e. “swoosh” to mix to suspend the gelatin in the thrombin solution. The sample is then left to sit for 5 min. Approximately 150 μL of the reconstituted test article is transferred to a TEG cup which is placed into the instrument. Immediately 210 μL of the blood anti-coagulated with 5 U/mL of heparin is added to the cup and quickly mixed. The TEG is then started and collects data for typically 20 minutes. The Amplitude (A) and Maximal Amplitude (MA) values were used to score product performance. Glutaraldehyde crosslinked gelatin (Glu-Gel) was used as a reference standard.

### Sample Preparation for TEG Testing:

Preparation of the “native” chitosan and glutaraldehyde crosslinked gelatin mixed with “native” chitosan:

Chitosan was purchased from the company Heppe Medical Chitosan. The product Chitosan 70/500 Lot 212-140211-01 was used which had a deacetylation degree of ˜70% and a molecular weight of ˜350 kD. In the following table 1 the exact amounts of glutaraldehyde crosslinked gelatin and chitosan that were weighed for the TEG testing are given. The glutaraldehyde crosslinked gelatin is made according to EP1803417B1.

TABLE 1
amounts of gelatin/chitosan used in TEG testing

| Glutaraldehyde | Chitosan | |
|---|---|---|
| crosslinked gelatin [g] | powder [g] | Sample Identification |
| 0.464 | 0.18 | Gelatin/Chitosan 30% (w/w solids) |
| | | Sample contains 30% (w/w solids) |
| | | of chitosan |
| 0.596 | 0.06 | Gelatin/Chitosan 10% |
| | | Sample contains 10% (w/w solids) |
| | | of chitosan |
| — | 0.5 g | Chitosan 100% |
| 0.777 | | Gelatin |

Preparation of the “Lyophilized” Chitosan and Glutaraldehyde Crosslinked Gelatin Mixed with “Lyophilized” Chitosan:

Chitosan was purchased from the company Heppe Medical Chitosan. The product Chitosan 70/500 Lot 212-140211-01 was used which had a deacetylation degree of ˜70% and a molecular weight of approx, 350 kD. The glutaraldehyde crosslinked gelatin is made according to EP1803417.

### Lyophilization Process to Obtain a Lyophilized Solid Chitosan Powder (Insoluble in Water)

For 2 l of 0.4% chitosan solution 8.0 g chitosan were weighed in a beaker and dissolved in dH₂O by addition of 2 M acetic acid to obtain solution with pH 4. A mass of 32.0 g mannitol was added as lyophilization aid. The prepared chitosan solution was lyophilized in a tray.

Freeze-dried chitosan was homogenized with a household hand blender and neutralized by incubation alongside ammonium hydroxide via the gas phase. Insoluble chitosan powder was washed with dH₂O to remove mannitol, rinsed in ethanol and dried under vacuum. In the following table 2 the exact amounts of glutaraldehyde crosslinked gelatin and chitosan that were weighed for the TEG testing are given.

TABLE 2
amounts of gelatin/chitosan used in TEG testing

| Glutaraldehyde | Chitosan | |
|---|---|---|
| crosslinked gelatin [g] | powder [g] | Sample Identification |
| 0.386 | 0.15 | Gelatin/Chitosan LYO 30% |
| | | Sample contains 30% (w/w solids) |
| | | of chitosan |
| | 0.4 | Chitosan LYO 100% |

All samples were tested after gamma irradiation. The samples Gelatin/Chitosan 30% and Chitosan 100% were also measured without performing a gamma radiation with a target dose of 35 Gy.

### TEG Test Results:

The results are depicted in FIG. 3 showing the test results of TEG measures with whole blood of glutaraldehyde crosslinked gelatin; lyophilized and native chitosan and mixtures thereof. All of the samples were gamma irradiated except if indicated otherwise.

### Discussion of TEG Results

The TEG results show that the chitosan samples as well as the mixtures between the glutaraldehyde crosslinked gelatin and the chitosan samples all formed a clot upon contact with whole blood that was either equally strong as the one obtained by the gelatin matrix or even stronger. Thus, this in vitro-parameter showed that chitosan or mixtures of glutaraldehyde crosslinked gelatin and chitosan hydrated with a thrombin solution are suitable variants for developing new hemostatic matrixes.

### Example 2.2

### In Vivo Results on Glutaraldehyde Crosslinked Gelatin-Chitosan Mixtures Hydrated with Thrombin Solution

Glutaraldehyde Crosslinked Gelatin—Lyophilized Chitosan Paste Hydrated with Thrombin

Chitosan was purchased from the company Heppe Medical Chitosan. The product Chitosan 70/100 Lot #212-070910-03 was used which had a deacetylation degree of ˜70%. and a molecular weight of approx. 200 kD. The glutaraldehyde crosslinked gelatin is made according to EP1803417.

Lyophilized chitosan powder was prepared as follows: 2 l of 0.4% chitosan solution were prepared by weighing 8.0 g Chitosan 70/100 in a beaker and an acetic acid solution (pH 4). 32.0 g mannitol was added as lyophilization aid. The prepared chitosan solution was lyophilized in a lyo tray.

Freeze-dried chitosan was homogenized with a household hand blender. Lyophilized chitosan powder was suspended in 500 ml ethanol and adjusted to a pH of 11 by addition of 1 M NaOH in ethanol. Insoluble chitosan powder was washed with distilled water to remove mannitol, rinsed in ethanol and dried under vacuum. For application, syringes were filled with 0.42 g crosslinked gelatin and 0.18 g lyophilized chitosan, resulting in a weight-percent ratio of 30% Chitosan, or 0.51 g crosslinked gelatin and 0.09 g lyophilized chitosan for a weight-percent ratio of 15%. The dry substances were each hydrated with 3 ml of a thrombin solution (500 IU/ml), which is equivalent to the thrombin solution according to EP1803417B1.

### Liver Punch Model:

All tests were performed on heparinized animals (pig). An excision lesion on the exposed lobe of the liver was made using biopsy punches (see FIG. 4).

Bleeding from the created lesion was assessed qualitatively (subjectively) according to the scale presented in FIG. 5; but not quantitatively (Bleeding score: 0: no bleeding (product saturated with blood); 1: ooze (blood out of product but no blood drop); 2: very mild (blood drop on the product); 3: mild (blood drop streams down); 4: moderate (small amount of blood streams down); 5: severe (large amount of blood streams down)).

Each lesion was topically treated with 1 ml product. Ringer-moistened gauze was used to help approximate the product to the lesion and the timer was started as soon as pressure was applied. The gauze was removed after 30 seconds and the timer was reset.

The degree of bleeding was assessed after 30 sec, and then approximately after 1, 2, 5 and 10 min. Product saturated with blood but without active bleeding was scored as 0. Saline solution was used to irrigate excess product from the lesion after the 5 minutes assessment.

### Results:

The final bleeding scores were further calculated to give per cent success rates. A success rate is achieved if the final bleeding score is below or at score 1 which is deemed to be clinically acceptable. A per cent success per time point over all samples was calculated and is shown in the diagram below. As a reference glutaraldehyde crosslinked gelatin made according to EP1803417B1 was used. As can be seen by the results the chitosan variants are at least equally suitable as hemostats in this test model as the commercially available glutaraldehyde crosslinked gelatin made according to EP1803417B1. The sample Gelatin/Chitosan 30% lyophilized did not perform as well as the gelatin sample after 600s, however sustained efficacy cannot be judged as no further time point was evaluated (see FIG. 6).

### Kidney Punch Model:

### Sample Preparation:

Chitosan was purchased from the company Heppe Medical Chitosan. The product Chitosan 70/500 Lot 212-140211-01 was used which had a deacetylation degree of ˜70% and a molecular weight of approx. 350 kD. The glutaraldehyde crosslinked gelatin is made according to EP1803417B1.

Syringes were filled with 0.42 g dry mass of glutaraldehyde crosslinked gelatin and 0.18 g chitosan. The final sample contained therefore 30% (w/w) chitosan. The sample was hydrated with 3.2 ml of a thrombin solution containing a concentration of 500 IU/ml thrombin and 40 mM of CaCl₂. All samples were gamma-irradiated with a target dose of 35 KGy.

### Pig Liver Punch Model:

### Sample Preparation and Liver Punch Model:

The sample preparation was made as described above for the other animal models; the liver punch model was performed as mentioned above.

### Results:

The results are depicted in FIG. 7, wherein hemostatic success over time in a liver punch model is shown. As can be seen, the chitosan containing paste showed at least an equal if not better performance than the crosslinked gelatin made according to EP1803417.

### Example 3

### Proof of Concept for the Formulation: Chitosan Plus PEG-SH Plus Chitosan Lactate Hydrated with a 40 mM Calcium Chloride Solution

### Proof of Concept Test for Adding PEG-SH and Chitosan Lactate to the Mixture of Glutaraldehyde Crosslinked Gelatin and Solid State Chitosan

### Sample Preparation:

Chitosan was purchased from the company Heppe Medical Chitosan. The product Chitosan 70/100 Lot #212-070910-03 was used which had a deacetylation degree of ˜70% and a molecular weight of approx. 200 kD. The glutaraldehyde crosslinked gelatin is made according to EP1803417B1. Chitosan lactate was also obtained from Heppe Medical Chitosan, PEG-SH was obtained from the company Baxter as it is a part of the commercially available product Coseal.

The solid component was prepared by mixing 0.5 g of chitosan. 0.22 g of PEG-SH and 0.077 g of chitosan lactate. All these components were mixed manually using a spatula. For hydration purposes 3 ml of a 40 mM CaCl₂ solution was used.

### Pig Liver Punch Model:

as explained in examples above

### Results:

Hemostasis was achieved (final score 1=oozing bleeding; n=1).

All patent filings, scientific journals, books, treatises, and other publications and materials discussed in this application are hereby incorporated by reference for all purposes. While exemplary embodiments have been described in some detail, by way of example and for clarity of understanding, those of skill in the art will recognize that a variety of modification, adaptations, and changes may be employed. Hence, the scope of the present invention should be limited solely by the claims.

1. A hemostatic composition comprising chitin or a water insoluble chitin derivative, in particulate form, wherein the composition is present in paste form.

2. The hemostatic composition according to claim 1, wherein the composition additionally comprises crosslinked gelatin.

3. The hemostatic composition according to claim 2, wherein the crosslinked gelatin is glutaraldehyde-crosslinked gelatin or genipin-crosslinked gelatin.

4. The hemostatic composition according to claim 1, wherein the chitin or the water insoluble chitin derivative is mixed with crosslinked gelatin in a ratio (in % (w/w)) of 5:95 to 80:20.

5. The hemostatic composition according to any one of claim 1, wherein the composition additionally comprises a biocompatible polymer suitable for use in hemostasis.

6. The hemostatic composition according to claim 5, wherein the biocompatible polymer suitable for use in hemostasis is a crosslinked protein, a crosslinked polysaccharide, a crosslinked biologic polymer, a crosslinked non-biologic polymer; or mixtures thereof.

7. The hemostatic composition according to claim 1 for use in the treatment of an injury selected from the group consisting of a wound, a hemorrhage, a damaged tissue, a bleeding tissue, and a bone defect.

8. A method of treating an injury selected from the group consisting of a wound, a hemorrhage, a damaged tissue and a bleeding tissue comprising administering a hemostatic composition according to claim 1 to the injury.

9. A kit for making a flowable paste of chitin or a water insoluble chitin derivative for the treatment of an injury selected from the group consisting of a wound, a hemorrhage, a damaged tissue, and bleeding tissue, comprising:
a) a dry hemostatic composition comprising chitin or a water insoluble chitin derivative, especially water insoluble chitosan, to be reconstituted to a flowable paste; and
b) a diluent for reconstitution of the hemostatic composition.

10. A method for providing a ready to use form of a hemostatic composition according to claim 1, wherein the hemostatic composition is provided in a first syringe and a diluent for reconstitution is provided in a second syringe, the first and the second syringe are connected to each other, and the fluid is brought into the first syringe to produce a flowable form of the hemostatic composition.

11. The hemostatic composition according to claim 1, wherein the water insoluble chitin derivative is water insoluble chitosan.

12. The hemostatic composition according to claim 3, wherein the crosslinked gelatin is type B gelatin.

13. The hemostatic composition according to claim 4, wherein the chitin or the water insoluble chitin derivative is mixed with crosslinked gelatin in a ratio (in % (w/w)) of 10:90 to 50:50.

14. The hemostatic composition according to claim 4, wherein the chitin or the water insoluble chitin derivative is mixed with crosslinked gelatin in a ratio (in % (w/w)) of 20:80 to 40:60.

15. The hemostatic composition according to claim 5, wherein the biocompatible polymer is selected from the group consisting of a protein, a polysaccharide, a biologic polymer, a non-biologic polymer; and derivatives and combinations thereof.
